(19) United States
(12) Patent Application Publication
(10) Pub. No.: US 2016/0106619 A1
(43) Pub. Date: Apr. 21, 2016

(54) METHOD AND EYEGLASS FOR THE RECOVERY AND REPAIR OF EYESIGHT

(71) Applicant: István MAGYAR, Szentendre (HU)

(72) Inventors: István MAGYAR, Szentendre (HU); Zoltán NAGY, Hajdúböszörmény (HU)

(73) Assignee: István Magyar, Szentendre (HU)

(21) Appl. No.: 14/890,113

(22) PCT Filed: May 7, 2014
(86) PCT No.: PCT/IB2014/061270

§ 371 Date: Nov. 9, 2015

(30) Foreign Application Priority Data
May 9, 2013 (HU) P1300298

(51) Int. Cl. A61H 5/00; G02C 7/16; G02C 7/02; G02C 7/10

(52) U.S. Cl. CPC A61H 5/00; G02C 7/101; G02C 7/104; G02C 7/16; G02C 7/02; A61H 2201/5028; A61H 2201/0157; A61H 2201/1207

(57) ABSTRACT

Method for the recovery and repair of eyesight through which out of the two eyes the weaker eye in need of repair or recovery is determined; both eyes are assigned an optical element influencing the passage of the incident light; the path of the light reaching the eye is periodically interrupted, thereby forcing the eye to perform additional focusing activity, so that transparency characteristics of the optical element assigned to the chosen or designated eye are left unchanged, and the transparency characteristics of the optical element assigned to the other eye are modulated so that the passage of incident light into the other eye is blocked and then unblocked with a frequency in the 0.2 Hz to 0.8 Hz range.

Corrective eyeglass composed of an eyeglass frame designed for holding and positioning the eyeglass on the head; containing two fixed optical elements influencing the passage of the received light, a control unit exerting temporal control over the optical characteristics of said optical element, and one power supply unit supplying at least one optical element and at least one control unit with electrical energy, wherein the optical element influencing the passage of incident light is embodied as a controlled light shutter, at least one control unit is electrically connected to the control unit, and said control unit is embodied as a control unit influencing the optical characteristics of at least one optical element based on a pre-defined controlling signal and providing asymmetric control with a frequency in the range of 0.2 Hz to 0.8 Hz to the optical elements.

### CROSS-REFERENCE TO RELATED APPLICATIONS

This application is the §371 National Stage of International Patent Application No. PCT/IB2014/061270, filed on May 7, 2014, which claims the benefit of Hungarian Patent Application No. P1300298, filed on May 9, 2013.

### FIELD OF THE INVENTION

The invention relates to a method and eyeglass for the recovery and repair of eyesight.

### BACKGROUND OF THE INVENTION

Human eyes participate unequally in the process of vision. The so-called dominant eye plays a more active role in focusing, while the non-dominant eye “only” follows the dominant eye and assists in spatial vision. Typically the vision of the non-dominant eye is weaker than that of the dominant eye. During childhood, in certain cases the non-dominant eye might become lazy accompanied by significant vision loss. Around middle age a slow but continuous vision loss begins. Typically the non-dominant eye suffers first from vision loss and degree of the loss is more pronounced. The individual does not register this vision loss for a long time, because the brain corrects for it as long as possible.

Numerous well-known methods and devices have been developed for the diagnosis and treatment of this process. U.S. Pat. No. 5,264,877 describes an eyeglass with lenses that are at least partially coated with a film that darkens in response to electricity to open and close the path of light in front of both eyes with a 4-12 Hz, or 6-15 Hz frequency, respectively. The human eye is unable to effectively sense these frequencies.

U.S. Pat. No. 5,452,026 describes a device and method containing one light-occluding unit in front of one or both eyes, and said units are powered in such a way that the device stays transparent to light for longer time in front of the weaker eye and for a shorter time in front of the stronger eye thus providing more time for the weaker eye to participate in vision. The device allows for the control of the duty cycle as well as the opening time of the light shutters in front of the eyes but the frequency also falls into the frequency range that is not sensed by the human eye.

U.S. Pat. No. 6,511,175 also describes and eyeglass containing two controllable light shutters with individually adjustable transparencies, which weaken the image in front of the stronger eye in a controllable manner thus impelling the weaker eye to work more efficiently.

The process described in U.S. Pat. No. 5,308,246 uses a single lens to simultaneously close, then simultaneously open the image in front of both eyes. Simultaneously opening and closing the light shutter in front of both eyes does not shift dominance to the weaker eye and is therefore not an efficient tool for vision repair or recovery.

The device described in U.S. Pat. No. 7,452,067 also contains to light shutters; however, certain areas of the lens do not darken but provide unimpeded vision. Instead of compelling the eye to re-focus, this design projects the image onto different areas of the fundus.

International publication No. WO/2013/040513 also proposes a bilateral periodic interruption of the image seen by the human eye, using frequencies in the range of 1 Hz to 15 Hz. Although sensed by the human eye, this interruption does not evoke a change in the brain that would result in improved vision.

### SUMMARY OF THE INVENTION

We have recognized that a significantly greater, and persistent effect can be achieved by simply forcing the lazier, weaker eye to take over dominance from the dominant eye from time to time and try to focus.

Based on our experiences it takes only a few minutes' training to significantly improve the vision of the non-dominant dye. Another very important factor is the length of the cycle. There have been many attempts at devising corrective eyeglasses that use a frequency not sensed by the human eye because its use is more comfortable. However, based on our experience, these frequencies are not effective. The reason is that if the human eye does not see the effect, it won't try to accommodate, therefore the training does not work. There have been attempts at using lower frequencies. The human eye senses these as vibrations but they are still too fast for the eye to allow easy accommodation. Other designs simultaneously shutter and then open the vision of both eyes. We do not consider these suitable for corrective treatment of the weaker eye, because this type of eyeglass does not compel the weaker eye to focus since after the opening it is the dominant eye that will automatically focus.

The invention concerns firstly a method for the recovery and repair of eyesight, by which the path of the light reaching the eye is periodically interrupted, thereby forcing the eye to perform additional focusing activity by inserting into the path of the incident light an optical element influencing the passage of the light and thus first achieving a temporary complete inhibition of the passage of the incident light, then completely freeing the path.

The invention concerns secondly a corrective eyeglass composed of a frame designed for holding and positioning the eyeglass on the head; at least one optical element inserted into the frame for the purpose of influencing the passage of the received light; at least one control unit exerting temporal control over the optical characteristics of at least one optical element; one power supply unit supplying at least one optical element and at least one control unit with electrical energy, where the control unit is electrically connected to the power supply unit and at least one optical element is electrically connected to the control unit, and the control unit is designed as a control unit using a pre-defined control signal to influence the transparency of at least one optical element.

The invention is detailed below in the form of an example of the embodiment and its mode of execution, with reference to the appended drawing wherein:

### BRIEF DESCRIPTION OF THE DRAWINGS

FIG. 1 is the flowchart of one of the possible modes of execution of the method according to the invention,

FIG. 2 is the schematic, exploded view of one of the embodiments of the eyeglass according to the invention, and

FIG. 3 shows the block diagram and connections of the possible electronics utilized in the eyeglass, as well as other operating parts and units of the eyeglass depicted in FIG. 2.

### DETAILED DESCRIPTION OF PREFERRED EMBODIMENTS

Hereinafter, only the preferred exemplary embodiments of the invention will be shown. It will be obvious to one skilled in the art that certain elements of the examples presented herein can be combined in different ways or modified or substituted without the resulting technical product falling outside the scope of the present invention.

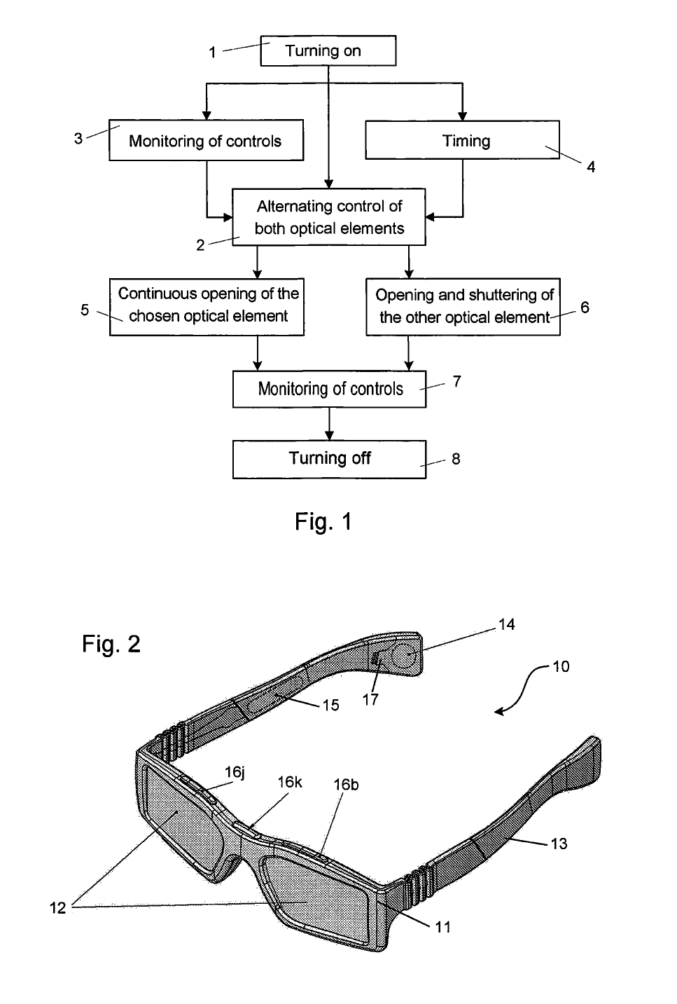

The proposed method in its most preferred embodiment is realized with the help of an eyeglass that, at first glance, looks traditional and is put on and worn by the user in the traditional manner; the structure and operation of this eyeglass will be described in a later section of this document. With respect to is operation, this eyeglass holds in front of the user's eyes two optical elements that can darken in response to the electrical control such as an impulse, and similarly, can become transparent again in response to another electrical control such as an impulse. As shown in FIG. 1, serving as an implementation of the proposed method, the operation of the electronic unit integrated into the eyeglass of the present example is started in Step 1 with the control associated with, or in the present case integrated into, the eyeglass. After starting, that is using the actuation, Step 1, to turn on the unit, the process prescribes an alternate darkening, Step 2, of the first optical element and then the second optical element, thus the user can alternately see with each eye his/her field of vision or the object, etc. contained therein that he/she is looking at. The alternating darkening, that is opening and shuttering of the optical elements is carried out at a frequency of 0.2-0.8 Hz, or as in the example shown, preferably at a frequency of 0.5 Hz in order to help the user realize that the vision in his/her eyes is unequal, or if his/her vision is very unequal the user can recognize this problem and can still obtain timely treatment for it.

This operating mode can be changed by the user in Step 3 via a control preferably also integrated into the eyeglass, or after a suitable period of time (such as 30 seconds to 1 minute) to enable the user to recognize the problem and experience a need for change, it can be changed via an automatic process in Step 4. Following upon Mode 1, in operating Mode 2 the user can begin the training of his/her weaker eye. The eye with the weaker vision is chosen with the help of a control, meaning that next the optical element assigned to the weaker eye is kept continuously open in Step 5, thus light can reach the user's eye uninterrupted, while the optical elements assigned to the stronger eye is periodically opened and shuttered in Step 6. The preferred frequency used in this step is identical to that of operating Mode 1, although this frequency is not compulsory because the shuttering and opening of the opening element can be carried out at other frequencies. In the case presented here, we use symmetrical control that is the open and shuttered periods are identical; however, the duty cycle can be modified as function of the characteristics of the user's eye and vision. For the desired mechanism of action it is important to keep the optical element in front of the weaker eye continuously open, while the optical element in front of the right-dominant-eye is preferably darkened every 2 seconds then made transparent again. When the optical elements in front of both eyes are open, the user keeps focusing with his/her dominant eye and only uses the non-dominant eye to follow the dominant one. When the optical element in front of the dominant eye is shuttered, the non-dominant eye becomes the dominant one and tries to focus. Unless and until the non-dominant eye is able to focus, the image seen by the user becomes blurred. Next, the optical element in front of the dominant eye is opened and the image seen by the user becomes sharp. Then the optical element in front of the dominant eye darkens again, and the non-dominant eye tries to focus again. By operating the appropriate control—this is monitored in Step 7—the user stops the operation of the eyeglass in Step 8 and thus completes the process.

FIG. 2 shows the schematic, exploded view of one of the embodiments of the eyeglass (10) according to the invention. The eyeglass (10) contains a frame (11) with standard temples. As a default, instead of regular glasses the frame contains the optical elements (12) mentioned above. In one embodiment of the invention the optical elements (12) may use products SF10084APT and SF10085APT of Shenzhen RIFDA LCD Co; their parameters are available in the product description for one skilled in the art. One or both temples (13) of the eyeglass (10) contain the control electronics and its power source (14) such as a button cell or button battery—indicated on the figure with a symbolic form and dashed line—, and the embodiment of the proposed method, the electronics (15) is connected to the optical elements (12), the controls (16) and the power source (14) via the electrical wires (not shown in the figure) running in the temple or temples (13) and the frame (11). In the case of the design shown in FIG. 2 control 16k serves as an on/off switch for the eyeglass (10) while controls 16b and 16j located above and assigned to each optical element (12) serve to choose the appropriate optical element (12). In the case presented here, controls 16k, 16b and 16j are set into the frame (11).

It is clear to one skilled in the art that the number and location of the controls (16) can be modified without changing the operation presented here. For example, by varying the length of time a control is pressed the user can activate different functions: turning on, turning off, switching between operating modes, and choosing the desired optical element. In the case presented here the user can turn on and off the device with the button utilized as control 16k; training of the left eye can be chosen with the button utilized as control 16b, in which case optical element 12 in front of the left eye is continuously transparent and the optical element in front of the right eye is turning on and off; and training of the right eye can be chosen with the button utilized as control 16j, in which case optical element 12 in front of the right eye is continuously transparent and the optical element (12) in front of the left eye is turning on and off.

A charger connection, not shown here, can be formed in the temple (13) to charge the button batteries (14) use as a power source, but a wireless inductive charging is also feasible such as that known in the area of mobile telephony. When using button cells, a lockable cell chamber (17) allowing replacement of the cells can be formed in the temple (13).

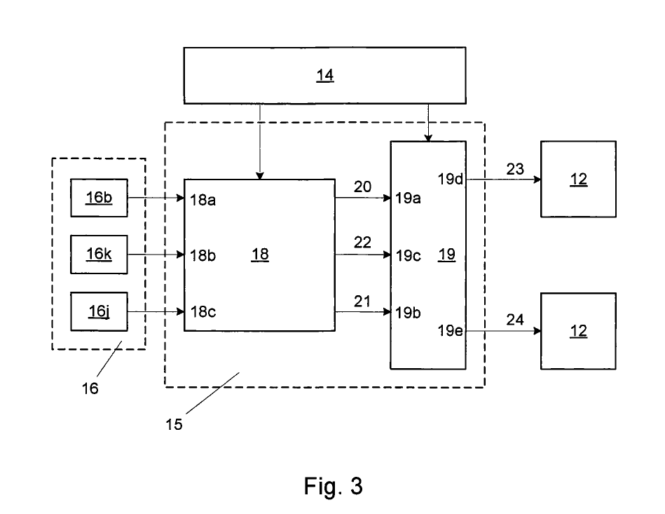

FIG. 3 shows the block diagram of one of the possible embodiments of the electronics used in the eyeglass shown in FIG. 2. The electronics contains an 8-bit low-power micro-controller (18) that receives user commands via control 16, controls the driver of the optical element 19, executes the appropriate timing, and is also responsible for the total energy usage of the eyeglass as well as low power utilization while the eyeglass is turned off. One skilled in the art can assuredly choose a micro-controller (18) suitable for such purpose from the current product range based on the specifications sheets of the manufacturers. Outputs of controls 16k, 16b and 16j of the eyeglass are connected to inputs 18a, 18b and 18c corresponding to this micro-controller 18. The actual connection depends on the type (opening, closing, switching) of the controls (16) and the input characteristics of the micro-controller (18). Outputs 18d, 18e and 18f of the micro-controller (18) are connected to inputs 19a, 19b and 19c of the driver of optical element 19 via leads 20, 21 and 22. Leads 20 and 21 are connected to inputs 19a and 19b of optical element state control of the driver of optical element 19; these inputs determine whether optical element 12 is darkened or transparent, and in the darkened state they determine the polarity of the alternating control voltage. Lead 22 connects to input 19c of enabling driver of optical element 19; micro-controller 18 enables or disables the operation of the driver of optical element 19.

The driver of optical element 19 transforms the 3 V battery voltage of power source 14 to the 10 V voltage necessary to shift the liquid crystal found in the (12) optical elements (the darkening of optical element 12), and contains the switches controlled by micro-controller 18 that transfer the transformed 10 V voltage to the appropriate optical element 12 with the correct polarity. As the device uses liquid crystals, the polarity of the control voltage must be flashed at a given frequency and using a method known to one skilled in the art, in order to prevent the overcharging and damaging of the crystal with direct current. This frequency must be significantly higher than the on/off frequency of optical element 12. Outputs 19d and 19e of optical element 19 are connected to the voltage input of optical element 12 via leads 23 and 24.

It is clear that the electronics required for the embodiment of the method are structurally simple, inexpensive to build, and safe to operate.

Although the specification refers to optical elements 12 arranged in eyeglass 10, it will be obvious that, as long as it does not prevent the embodiment of the proposed method, prescription lenses tailored to the obligatory correction of eye disorders of the user can also be used with optical elements 12. These lenses may be placed in front of or behind the optical elements 12; their position may be determined on the basis of optical considerations.

Said lenses can also be devised as sunglass lenses, or instead of prescription lenses, sunglass lenses or other corrective lenses may be used depending on the actual embodiment.

### LIST OF REFERENCE NUMBERS

1-8: Steps
10: Eyeglass
12: Optical element
13: Temple
14: Power Source
15: Electronics
16, 16k, 16b and 16j: Controls
17: Battery chamber
18: Micro-controller
18a, 18b and 18c: Inputs
18d, 18e and 18f: Outputs
19: Optical element driver
19a, 19b and 19c: Inputs
19d and 19e: Outputs
20-24: Leads

1-19. (canceled)

20. A method for the recovery and repair of eyesight, comprising:
periodically interrupting the path of the light reaching the eye, thereby forcing the eye to perform additional focusing activity;
wherein periodically interrupting the path of the light reaching the eye includes inserting into the path of the incident light an optical element influencing the passage of the light and thus first achieving a temporary complete inhibition of the passage of the incident light, then completely freeing the path;
wherein both eyes are assigned an optical element influencing the passage of the incident light;
wherein out of the two eyes the weaker eye in need of repair or recovery is chosen; and
wherein transparency characteristics of the optical element assigned to the chosen eye are left unchanged, and the transparency characteristics of the optical element assigned to the other eye are modulated so that the passage of incident light into the other eye is blocked and then unblocked with a frequency in the 0.2 Hz to 0.8 Hz range.

21. The method according to claim 20, wherein both eyes are assigned identical optical elements.

22. The method according to claim 20, wherein the passage of incident light into the other eye is blocked and then unblocked with a frequency of 0.5 Hz.

23. The method according to claim 20, wherein, at the stage where out of the two eyes the weaker eye in need of repair or recovery is chosen, the transparency characteristics of both optical elements assigned to each eye are modulated in an alternating but coordinated manner.

24. The method according to a claim 20, wherein the passage of incident light into the other eye is blocked for a pre-defined period of time with the optical element assigned to the other eye.

25. The method according to claim 24, wherein said period of time is chosen from the range of 2 minutes to 30 minutes.

26. An eyeglass for the recovery and repair of eyesight, the eyeglass comprising:
an eyeglass frame designed for holding and positioning the eyeglass on the head;
at least one optical element inserted into the frame for the purpose of influencing the passage of the received light;
at least one control unit exerting temporal control over the optical characteristics of at least one optical element; and
one power supply unit supplying at least one optical element and at least one control unit with electrical energy;
wherein the control unit is electrically connected to the power supply unit;
wherein the at least one optical element influencing the passage of incident light includes a controlled light shutter,
wherein the at least one optical element is electrically connected to the control unit;
wherein the control unit is embodied as a control unit influencing the optical characteristics of at least one optical element based on a pre-defined controlling signal;
wherein the eyeglass frame contains two fixed optical elements influencing the passage of the received light;
wherein the control unit is configured to provide asymmetric control with a frequency in the range of 0.2 Hz to 0.8 Hz to the optical elements.

27. The eyeglass according to claim 26, wherein the at least one optical element including a controllable light shutter is a liquid crystal (LCD) light shutter.

28. The eyeglass according to claim 26, wherein the control unit is assigned a start switch.

29. The eyeglass according to claim 28, wherein there are two start switches assigned to the control unit.

30. The eyeglass according to claim 28 wherein the start switch is a push-button.

31. The eyeglass according to claim 28, wherein the start switch is integrated into the eyeglass frame.

32. The eyeglass according to claim 29, wherein the start switches are connected to each of the optical elements of the eyeglass, each of the optical elements including controllable light shutters.

33. The eyeglass according to claim 28, wherein the control unit is integrated into one of the eyeglass frames or temples.

34. The eyeglass according to claim 28, wherein the power supply unit is a replaceable cell or rechargeable accumulator integrated into at least one of the eyeglass frames or temples.

35. The eyeglass according to claim 28, wherein the power supply unit is a rechargeable accumulator integrated into at least one of the eyeglass frames or temples in a sealed and closed chamber.

36. The eyeglass according to claim 29, characterized in that the control unit is assigned a central start switch, and each optical element of the eyeglass includes a controllable light shutter and is assigned a selector switch.

37. The eyeglass according to claim 26, wherein the optical element including the controllable light shutter is assigned a prescription lens.

38. The eyeglass according to claim 26, wherein the optical element including the controllable light shutter is, itself, a corrective lens.
